(19) United States
(12) Patent Application Publication
(10) Pub. No.: US 2018/0296608 A1
(43) Pub. Date: Oct. 18, 2018

(54) Compositions containing SPHEROID CELL AGGREGATES for enhance ovary function and preparation method of the same

(71) Applicants: COLLEGE OF MEDICINE POCHON CHA UNIVERSITY INDUSTRY-ACADEMIC COOPERATION FOUNDATION, Rocheon-si (KR); KOREA UNIVERSITY RESEARCH AND BUSINESS FOUNDATION, Seoul (KR)

(72) Inventors: Tae-Hee KIM, Bucheon-si (KR); SangHoon LEE, Seoul (KR); Gi Jin KIM, Seongnam-si (KR); Ji Young HWANG, Seoul (KR); Jong Ho CHOI, Yongin-si (KR); Yesl JUN, Seoul (KR)

(21) Appl. No.: 15/764,937

(22) PCT Filed: Sep. 27, 2016
(86) PCT No.: PCT/KR2016/010828

§ 371 Date: Mar. 30, 2018

(30) Foreign Application Priority Data
Oct. 2, 2015 (KR) 10-2015-0139287

(51) Int. Cl. A61K 35/50; A61P 15/08; A61K 9/00; C12N 5/0775

(52) U.S. Cl. CPC A61K 35/50; C12N 5/0668; A61K 9/0019; A61P 15/08

(57) ABSTRACT

The present invention can improve or recover ovarian functions of mammals by containing spheroidal cell aggregates including placenta-derived mesenchymal stem cells as an active ingredient.

### TECHNICAL FIELD

The present invention relates to a pharmaceutical composition containing spheroidal cell aggregates for improving ovarian functions and a method of preparing the same and more particularly to a pharmaceutical composition which is applicable to relieving or treating early ovarian failure, infertility/subfertility, early menopausal, menopausal and climacteric symptoms by producing placenta-derived mesenchymal stem cells in the form of spheroidal cell aggregates.

### BACKGROUND ART

The ovaries are organs which play an important role in maintaining the quality of life of women by not only maintaining the health of the female reproductive system, but also maintaining the balance of the hormone production system. Menopause resulting from ovarian dysfunction or aging can lead to many women's systemic problems (such as dementia, osteoporosis, heart disease, climacteric disorders and metabolic disorders).

Climacteric disorders are due to falling levels of estrogen, which is a female hormone that performs various functions such as helping with blood circulation, weight control and bone management as well as maintaining female sexual functions. Climacteric disorders happen mainly during climacterium at the age of about 50. However, identical symptoms are observed in estrogen deficient patients as well due to other causes such as ovariectomy and ovarian dysfunction. These climacteric diseases are often due to menopause caused by dysfunction or aging of the ovary which plays an important role in the balance of the hormone production system of women.

Specific symptoms of climacteric diseases include physical symptoms such as climacteric osteoporosis, facial flushing, abdominal obesity, cervical atrophy, cognitive disorders, Alzheimer's disease, stagnant flow, hyperhidrosis and skin aging, as well as psychoneural symptoms such as depression, difficulty concentrating, insomnia, headache, tinnitus and hypersensitivity. Relieving climacteric symptoms and maintaining female hormone balance are essential to maintaining the life quality of women.

Many studies reported that some herbs and foods do not prevent climacteric symptoms, but alleviate clinical symptoms. Nonetheless, there is no method of treating premature ovarian failure and menopause to date. Despite the risk of developing breast cancer, hormone replacement therapy is the only treatment method. For this reason, postmenopausal women live without reproductive hormones such as estrogen or progesterone for half their lives. There is a need for a fundamental solution to this.

Stem cell-based therapies are therapeutic approaches which have recently received much attention in the fields of autoimmune diseases, regenerative medicine and tissue engineering. Stem cell-based therapies are applied to immunotherapy for bone or cartilage regeneration and chemotherapy as well as clinically for therapeutic purposes of urinary incontinence, type 1 diabetes, cardiomyopathy, complications of Crohn's disease and the like. However, stem cell-based therapies have not been widely applied to other clinical diseases and basic research thereon is still actively underway.

The placenta is an organ involved in the synthesis and secretion of cytokines and proteins that have many kinds of inherent physiological activities as well as serve to supply nutrients and as carriers of wastes and oxygen, which are essential for fetal development during pregnancy. The placenta is discharged from the uterus upon childbirth. The placenta has been recognized as a temporary organ which is disused after delivery. However, in recent years, various cells such as mesenchymal cells, decidua cells, amnion and endothelial cells can be recovered depending on placental sites, and research including the characterization of these cells and research on therapeutic efficacy in various degenerative diseases are underway.

### PRIOR ART LIST

Korean Patent No. 10-1282926, granted on Jul. 1, 2013.
Korean Patent No. 10-0900309, granted on Jun. 2, 2009.

### DISCLOSURE

### Technical Problem

Therefore, the present invention has been made in view of the above problems, and it is an object of the present invention to provide a method of producing placenta-derived mesenchymal stem cells in the form of spheroidal cell aggregates and a pharmaceutical composition for improving ovarian functions which contains the spheroidal cell aggregates as an active ingredient and is thus applicable to relieving or treating early ovarian failure, infertility/subfertility, early menopausal, menopausal and climacteric symptoms.

### Technical Solution

This application claims the benefit of priority to Korean Patent Application No. 10-2015-0139287 filed on Oct. 2, 2015, the entire contents of which are incorporated herein by reference.

In accordance with the present invention, the above and other objects can be accomplished by the provision of a pharmaceutical composition for improving ovarian functions containing, as an active ingredient, spheroidal cell aggregates including placenta-derived mesenchymal stem cells.

The spheroidal cell aggregates may have less junction boundary between cells due to junctional complexes between adjacent cells and extracellular matrices (ECMs) and thus a relatively smooth outer surface.

The pharmaceutical composition for improving ovarian functions may be used to relieve or treat at least one symptom selected from the group consisting of early ovarian failure, infertility/subfertility, early menopausal, menopausal and climacteric symptoms.

The pharmaceutical composition for improving ovarian functions can facilitate expression of any one protein selected from the group consisting of Nobox, Nanos3, Lhx8 and a combination thereof.

The pharmaceutical composition for improving ovarian functions can increase the number of ovarian follicles.

The pharmaceutical composition for improving ovarian functions can improve internal estradiol levels.

The pharmaceutical composition for improving ovarian functions may further include an adjuvant.

The adjuvant may include saline, phosphate buffered saline (PBS), a medium or mineral oil. In addition, the pharmaceutical composition for improving ovarian functions including an adjuvant may include the active ingredient in an amount of 0.1 to 99% by weight, or 20 to 80% by weight.

The pharmaceutical composition for improving ovarian functions may be formulated in the form of an injection.

In accordance with another aspect of the present invention, there is provided a use for preparing a pharmaceutical composition for improving ovarian functions including spheroidal cell aggregates including placenta-derived mesenchymal stem cells.

The spheroidal cell aggregates may be obtained by three-dimensionally culturing placenta-derived mesenchymal stem cells and may have less junction boundary between cells due to junctional complexes formed between adjacent cells and extracellular matrices (ECMs), and thus a relatively smooth outer surface, compared to placenta-derived mesenchymal stem cells before culture.

The spheroidal cell aggregates may have a mean diameter of 250 μm or less, preferably 100 to 250 μm.

The use for preparation of the pharmaceutical composition for improving ovarian functions may include relieving or treating at least one symptom selected from the group consisting of early ovarian failure, infertility, subfertility, early menopausal, menopausal and climacteric symptoms.

Regarding the use for preparation of the pharmaceutical composition for improving ovarian functions, the pharmaceutical composition for improving ovarian functions may further include an adjuvant, in addition to the spheroidal cell aggregates. The adjuvant may include saline, phosphate buffered saline (PBS), a medium, or mineral oil. In addition, the pharmaceutical composition for improving ovarian functions including an adjuvant may include the active ingredient in an amount of 0.1 to 99% by weight, preferably 20 to 80% by weight. In addition, regarding the use for preparation of the pharmaceutical composition for improving ovarian functions, the pharmaceutical composition for improving ovarian functions may be formulated in the form of an injection.

Regarding the use for preparation of the pharmaceutical composition for improving ovarian functions, the pharmaceutical composition for improving ovarian functions can facilitate expression of any one protein selected from the group consisting of Nobox, Nanos3, Lhx8 and a combination thereof.

Regarding the use for preparation of the pharmaceutical composition for improving ovarian functions, the pharmaceutical composition for improving ovarian functions can increase the number of ovarian follicles.

Regarding the use for preparation of the pharmaceutical composition for improving ovarian functions, the pharmaceutical composition for improving ovarian functions can improve internal estradiol levels.

In accordance with another aspect of the present invention, there is provided a use of a pharmaceutical composition for improving ovarian functions including improving ovarian functions by containing spheroidal cell aggregates including placenta-derived mesenchymal stem cells.

In accordance with another aspect of the present invention, there is provided a method of producing spheroidal cell aggregates for improving ovarian functions including: adding placenta-derived mesenchymal stem cells to concave microwells in a concave microwell plate; culturing the placenta-derived mesenchymal stem cells in the microwells using a medium for culturing cell aggregates containing a cell growth factor to create spheroidal cell aggregates including placenta-derived mesenchymal stem cells; and harvesting the spheroidal cell aggregates.

In accordance with another aspect of the present invention, there is provided a method of preventing or treating a disease relating to deterioration in ovarian functions including administering, to a mammal, a pharmaceutical composition for improving ovarian functions containing spheroidal cell aggregates including placenta-derived mesenchymal stem cells. The spheroidal cell aggregates are used as an active ingredient of the pharmaceutical composition described above.

The spheroidal cell aggregates may have less junction boundary between cells due to junctional complexes between adjacent cells and extracellular matrices (ECMs), and thus a relatively smooth outer surface.

The spheroidal cell aggregates may be obtained by three-dimensionally culturing placenta-derived mesenchymal stem cells and may have less junction boundary between cells due to junctional complexes formed between adjacent cells and extracellular matrices (ECMs), and thus a smooth outer surface, compared to placenta-derived mesenchymal stem cells before culture.

The spheroidal cell aggregates may be used in combination with an adjuvant. The adjuvant may include saline, phosphate buffered saline (PBS), a medium or mineral oil.

The pharmaceutical composition for improving ovarian functions including an adjuvant may include the active ingredient in an amount of 0.1 to 99% by weight, preferably 20 to 80% by weight. In addition, the pharmaceutical composition for improving ovarian functions may be formulated in the form of an injection.

The disease relating to deterioration in ovarian functions may include at least one symptom selected from the group consisting of early ovarian failure, infertility, subfertility, early menopausal, menopausal and climacteric symptoms.

The method of preventing or treating a disease relating to deterioration in ovarian functions may include injecting the pharmaceutical composition for improving ovarian functions into the female genital organ or an organ adjacent thereto. Examples of the female genital organ or the organ adjacent thereto may include the ovaries, the fallopian tubes, the abdominal cavity, or the peritoneum.

Regarding the method of preventing or treating a disease relating to deterioration in ovarian functions, the pharmaceutical composition for improving ovarian functions can facilitate expression of any one protein selected from the group consisting of Nobox, Nanos3, Lhx8 and a combination thereof.

Regarding the method of preventing or treating a disease relating to deterioration in ovarian functions, the pharmaceutical composition for improving ovarian functions can increase the number of ovarian follicles when injected into the body.

Regarding the method of preventing or treating a disease relating to deterioration in ovarian functions, the pharmaceutical composition for improving ovarian functions can improve internal estradiol levels when injected into the body.

Hereinafter, the present invention will be described in more detail.

As used herein, numbers, which are used with or without the terms relating to a degree, such as “about”, “substantially”, “comparatively” and “relatively”, include the aforementioned meaning and the range close to the numerical value including an error allowed by inherent properties. In addition, the terms relating to a degree such as “about”, “substantially”, “comparatively” and “relatively” are used to avoid interpretation of the content mentioned in this specification in an unreasonably narrow sense due to exact or absolute numbers or expressions from and to inappropriately prevent an infringer from using this narrow interpretation.

Singular forms are intended to include both singular forms and plural forms, unless context clearly indicates otherwise.

As used herein, the term “menopausal or climacteric and disease” refers to one of the following systems: i) symptoms due to vascular changes; ii) symptoms due to musculoskeletal changes; iii) symptoms due to genitourinary changes; iv) symptoms due to changes in the cranial nervous system; and v) symptoms due to general changes, which result from deterioration in secretion of estrogen (female hormone) due to ovarian dysfunction or aging, or a disease derived therefrom. The main symptoms due to vascular changes include facial flushing, tachycardia, perspiration, headache and the like, the symptoms due to musculoskeletal changes include muscle pain, joint pain, back pain and the like, the symptoms due to genitourinary changes include frequent urination, incontinence and the like, the symptoms due to changes in the cranial nervous system include memory loss, depression, difficulty concentrating, dizziness and the like, and the symptoms due to general changes include amblyopia and changes in skin and hair.

As used herein, the term “placenta” refers to in vivo tissues or placenta derivatives made for the fetus during pregnancy and sub-tissues thereof include the chorionic membrane (CM), the chorionic membrane and chorionic trophoblast layer (CMT), the total chorionic trophoblast layer (tCT), the upper portion of the chorionic trophoblast layer (uCT) and the basal portion of the chorionic trophoblast layer (bCT). In addition, the placenta may be any placenta of a mammal, for example, placenta of human or pig, but the present invention is not limited thereto.

In order to accomplish the object, the pharmaceutical composition for improving ovarian functions according to one embodiment includes cell aggregates containing placenta-derived mesenchymal stem cells (hereinafter referred to as “PD-MSCs”) as an active ingredient.

Unlike other mesenchymal stem cells such as bone marrow-derived mesenchymal stem cells (hereinafter, referred to as “BM-MSCs”) and adipose-derived mesenchymal stem cells (adipose-derived MSCs), placenta-derived mesenchymal stem cells (PD-MSCs) have an advantage of securing a number of cells in a relatively easy way without an invasive process, i.e., isolation from the placenta.

Like bone marrow-derived mesenchymal stem cells (BM-MSCs), placenta-derived mesenchymal stem cells (PD-MSCs) have multipotency, which is the ability to differentiate into mesodermal lineages, in particular, into oocyte-like cells, including adipogenic, chondrogenic and osteogenic abilities. Furthermore, the placenta-derived mesenchymal stem cells are reported to have great effects on controlling immunity relating to human leukocyte antigen-G (HLA-G).

In the present invention, the placenta-derived mesenchymal stem cells are used in the form of cell aggregates for the purpose of improving ovarian functions. In this case, the cell aggregates are three-dimensionally cell-cultured to have a predetermined volume while including placenta-derived mesenchymal stem cells, rather than monolayer culture generally used as cell culture, and this type of cell aggregates is contained as an active ingredient of the pharmaceutical composition for improving ovarian functions.

Three-dimensional cell culture is a cell culture method which compensates for the drawbacks of typical monolayer culture (2D culture), that is, limitation in reproduction of the complicated microenvironment of biosystems, and provides a similar environment to the in vivo environment which is maintained by the cell-cell and cell-extracellular matrix (cell-ECM).

Compared to mesenchymal stem cells adhesion-cultured as a monolayer, three-dimensionally cultured products of mesenchymal stem cells are self-activated to increase expression of prostaglandin E2 genes, which facilitate expression of C-X-C chemokine receptor type 4, IL-24 or tumor necrosis factor-inducible gene 6 protein and adhesion of endothelial cells, and thereby enhance anti-inflammatory or anti-cancer activities.

The cell aggregates including placenta-derived mesenchymal stem cells include junctional complexes and extracellular matrices (ECMs) formed between adjacent cells, so that they can have an outer surface with a relative smoothness (unevenness) due to less junction boundary between cells resulting from them. The cell aggregates may be made of placenta-derived mesenchymal stem cells, and junctions and cell-extracellular matrices thereof.

The cell aggregates may have a three-dimensionally spheroidal structure. The term “spheroidal”, as used herein, means a three-dimensional structure wherein cells are aggregated such that they have a substantially circular or oval cross-sectional surface, and it is obvious to those skilled in the art that this form should be determined in consideration of properties of cells or cell aggregates, or does not mean a completely spheroidal or oval shape.

The cell aggregates may be produced using a method for three-dimensionally culturing cell aggregates such as hanging drop culture, spinner flask culture, or three-dimensional cell culture using a concave microwell plate.

The cell aggregates derived from monolayer-cultured cells have a structure in which cells are agglomerated together and connected by cell junction, in an early culture phase, but have an overall uneven outer appearance while allowing the shapes of respective cells to be exposed to the surfaces of cell aggregates, and have an overall larger diameter than three-dimensionally cultured cell aggregates.

As three-dimensional culture proceeds, the diameter of cell aggregates decreases and then maintains a predetermined level. At this time, uneven portions of cell aggregates on the surfaces thereof gradually change to be relatively smooth and form a spheroidal three-dimensional shape which is mentioned throughout the present invention.

The cell aggregates can be applied to pharmaceutical compositions so long as they have a size enabling cells in cell aggregates to maintain their activity and three-dimensional structures, specifically the cell aggregates may have a diameter of 250 μm or less, 100 to 250 μm. By using cell aggregates having a diameter within this range, it is possible to form cell aggregates which maintain the overall activity and stability of cells in cell aggregates and contain junctional complexes and cell-extracellular matrices in a degree suitable for application to the pharmaceutical composition for improving ovarian functions, using placenta-derived mesenchymal stem cells.

The pharmaceutical composition is used to improve ovarian functions of mammals as well as to relieve or treat early ovarian failure, early menopause, menopause, infertility/subfertility and climacteric symptoms.

During oogenesis of mammals, germ lines interact with ovarian follicles. Recovery of ovarian functions in reproductively mature mammals includes generation of new oocytes and primordial follicles. In this case, marker proteins relating to oogenesis such as Nobox, Nanos3 and Lhx8, and ovarian folliculogenesis are expressed.

Nobox gene expression in the ovaries is essential to formation and maintenance of primordial follicles. Nanos derived from RNA binding proteins relating to germ cell development has high preservability and is thus utilized as a biomarker useful for recovery or maintenance of ovarian functions. Lhx8 is a protein which is mainly generated in reproductive cells and induces LIM-homeobox transcription factors essential for oogenesis in mammals.

In the ovarian follicles microenvironment, ovarian recovery is reported to be induced by expression of Nobox, Nanos3 and Lhh8.

The present inventors found that, when the pharmaceutical composition is administered to unilaterally ovariectomized (½ ovariectomized) rat models, Nobox, Nanos3 and Lhh8 are expressed and thus identified that the pharmaceutical composition has a potency of recovering ovarian functions of women.

When the pharmaceutical composition containing cell aggregates as an active ingredient is administered to female mammals, it activates the ovarian follicle microenvironment to provide therapeutic effects of restoring ovarian functions and increase the number of ovarian follicles.

Administration of the pharmaceutical composition increases internal estradiol levels and induces ovarian folliculogenesis, when ovarian functions are deteriorated for reasons such as unilateral ovariectomy.

These results show that administration of the pharmaceutical composition provides recovery and regeneration of dysfunctional ovaries through oogenesis based on folliculogenesis.

The pharmaceutical composition contains the aforementioned cell aggregates as an active ingredient. This case exhibits a longer period of time at which cell activity of cell aggregates is maintained under ovarian follicles microenvironment and is more effective in increasing the amount of expressed oogenesis markers as well as the number of ovarian follicles, compared to the case where the pharmaceutical composition contains monolayer-cultured cells as an active ingredient. That is, the pharmaceutical composition has a higher therapeutic potential in terms of improvement of ovarian functions than when monolayer-cultured cells are used.

Premature ovarian failure and early menopause lead to hormone deficiency and are accompanied by mortality, coronary heart disease, dementia, osteoporosis, psychological changes such as senile depression, sexual dysfunction, atrophic vaginitis and the like. In addition, menopausal symptoms may include irregular menstrual cycles, facial flushing, sweating, insomnia, vaginal dryness, urinary incontinence, hypoactive sexual desire disorder, osteoporosis and the like.

Hormone replacement therapy is used as a prescription to alleviate climacteric symptoms and is known as almost the only treatment at the moment. However, short-term prescription is mainly used since it is reported that there is a possibility that complications such as breast cancer may occur when hormone treatment after menopause is applied for a long period of time.

Unlike conventional hormone replacement therapy, the pharmaceutical composition can provide an effect of maintaining estradiol levels, for example, caused by recovery of ovarian functions by adding spheroidal cell aggregates including placenta-derived mesenchymal stem cells. This effect can also lead to effects including high stability, maintenance of healthy conditions during menopause, minimized complications in senescence and minimized morbidity which are purposes for climacteric treatment.

In addition, the pharmaceutical composition can improve quality of life of women and reduce medical costs caused by development of aging-related diseases through recovery of ovarian functions and extended ovarian functional cycles.

The pharmaceutical composition of the present invention contains the fore-mentioned spheroidal cell aggregates including placenta-derived mesenchymal stem cells as an active ingredient. In addition, the pharmaceutical composition of the present invention may further include a pharmaceutically acceptable carrier (or adjuvant). Any carrier or adjuvant may be used as the carrier (or adjuvant) without limitation so long as it is commonly used in cell therapy. Specifically, the pharmaceutically acceptable carrier may include a sterilized solution (for example, physiological saline), a non-aqueous solvent or the like. In addition, if necessary, a stabilizing agent, an adjuvant (for example, complete Freund's adjuvant, incomplete Freund's adjuvant or the like), an isotonic agent, a preservative or the like may be used.

The pharmaceutical composition of the present invention may be formulated in the form of an injection, preferably, an injection for the ovary (or uterus) administration, by an ordinary preparation method.

The administration amount of spheroidal cell aggregates including placenta-derived mesenchymal stem cells contained as an active ingredient in the pharmaceutical composition of the present invention can be changed depending on patient conditions and weight, severity of disease, drug type, and route and duration of administration, and can be suitably selected by those skilled in the art. For example, the spheroidal cell aggregates may be administrated in an amount of about 500 units/kg, based on the number of cell aggregates, and administered once or several times repeatedly, if necessary. In addition, the spheroidal cell aggregates may be administered at a dose of 5×10⁴ cells/kg to 1×10⁶ cells/kg, based on the number of cells.

The pharmaceutical composition may be applied by oral, rectal, intravenous, nasal, peritoneal, subcutaneous or topical administration, specifically, topical administration through direct injection into the uterine or ovarian tissues, but the present invention is not limited thereto.

In accordance with another aspect of the present invention, there is provided a method of producing spheroidal cell aggregates for improving ovarian functions including: adding monolayer-cultured placenta-derived mesenchymal stem cells to a concave microwell plate for culturing cell aggregates; culturing the placenta-derived mesenchymal stem cells in the concave microwell using a medium for culturing cell aggregates containing a cell growth factor to create spheroidal cell aggregates including placenta-derived mesenchymal stem cells; and harvesting the spheroidal cell aggregates.

The microwell may be applied so long as it has a shape suitable for culturing cell aggregates, but, for example, the concave microwell plate shown in FIG. 1 may be applied and may be manufactured using soft-lithography and meniscus of prepolymers or the like.

The concave microwell plate is a means for mass-producing micro-sized spheroidal cell aggregates that have a controlled size and shape, and enable the added cells to have a self-assembled three-dimensional structure without using an additional device or labor for culturing cell aggregates.

The concave microwell plate may be made of silicone, for example, polydimethylsiloxane (PDMS).

Regarding the diameter and depth of microwells, a predetermined shape and size may be applied depending on properties of cells administered to the microwells, for example, and the diameter of the microwells (shown as φ in FIG. 1) may be 300 to 1,000 μm, more specifically, 400 to 600 μm.

The microwell having a concave structure may be produced in the form of a mold having dozens to several thousand microwells formed thereon. The mold applied to the present invention shown in FIG. 1 is a multiple-concave mold which allows formation of spheroidal cell aggregates and is thus optimal for cell growth, and has an advantage of simultaneously producing several hundred to several thousand spheroidal cell aggregates.

The microwell for culturing spheroidal cell aggregates may be coated to prevent cell adhesion on the concave surface of wells before cell administration. For example, the surface of well for culturing cell aggregates may be coated with bovine serum albumin.

It is more effective to use plates having a concave microwell array formed thereon as the wells for culturing cell aggregates. In order for administered (seeded) cells to be effectively formed into spheroidal cell aggregates, cells are administered in a predetermined amount in accordance with the concave shape of the mold and are then cultured under certain culture conditions, so that cell aggregates with a substantially uniform size can be obtained in a relatively simple way.

The three-dimensional cell culture using concave microwells is advantageously a relatively simple process because it enables cell aggregates having a three-dimensional shape to be formed by a self-assembly process of added cells. In addition, the three-dimensional cell culture has advantages of being capable of adjusting the size of cell aggregates by controlling the diameter and depth of concave wells, of producing cell aggregates including placenta-derived mesenchymal stem cells, cell junctions to mutually connect these cells and extracellular matrix materials using monolayer-cultured placenta-derived mesenchymal stem cells, and of obtaining cell aggregates having an intended size, and a substantially uniform size and shape.

The placenta-derived mesenchymal stem cells added to microwells can be obtained by treating cells derived from the placenta and then culturing. At this time, the treatment process may be any well-known method of obtaining placenta-derived mesenchymal stem cells. Specifically, the placenta-derived mesenchymal stem cells may be obtained by removing the membrane of the chorionic plate from the human placenta and monolayer-culturing the cells scrapped from the removed membrane.

The addition may be carried out by locating cells in hemispheric concave areas of microwells depending on the size of predetermined microwells in consideration of the intended size of the cell aggregates. At this time, the number of used cells may be determined at a substantially predetermined level in consideration of the intended size of cell aggregates or the intended size of the microwells.

The added cells are cultured using a medium for culturing cell aggregates after checking whether or not cells sink well in the microwells (culture step).

The medium for culturing cell aggregates may include as, growth factors, fibroblast growth factors (FGFs) and heparin. The culture step may be carried out while changing a culture solution every 2 to 3 days.

The culture step may be carried out for 1 to 7 days, preferably 2 to 5 days and the culture step may be carried out to an extent that cell aggregates having a relatively smooth outer surface and a spheroidal shape according to the present invention, that is, cell aggregates where junctions and extracellular matrices between placenta-derived mesenchymal stem cells are sufficiently formed.

The harvest step is to collect cell aggregates containing placenta-derived mesenchymal stem cells produced by the aforementioned process. The size of harvested cell aggregates can be adjusted by controlling the diameter and depth of the microwells and cell aggregates with a substantially uniform size can be produced and harvested.

For example, when cell aggregates are produced using microwells having a mean diameter of about 500 μm, spheroidal cell aggregates, which are being self-assembled immediately after administration, have a diameter of about 250 μm, and as culturing proceeds, spheroidal cell aggregates, which are finished being formed to have a relatively smooth outer appearance, have a diameter of about 150 μm.

For example, when cell aggregates are produced using microwells having a mean diameter of about 300 μm, spheroidal cell aggregates, which are being self-assembled immediately after administration, have a diameter of about 100 μm, and as culturing proceeds, spheroidal cell aggregates, which are completely formed to have a relatively smooth outer appearance, have a diameter of about 80 μm.

The method of producing spheroidal cell aggregates for improving ovarian functions enables placenta-derived mesenchymal stem cells to be produced in the form of cell aggregates having a substantially uniform diameter by a relatively simple method, and production of cell aggregates, where junctions between cells and extracellular matrices are sufficiently formed during culture, which are applicable as an active ingredient of the pharmaceutical composition for improving ovarian functions. The method of producing cell aggregates for improving ovarian functions is applicable to the production of the pharmaceutical composition for improving ovarian functions.

### Advantageous Effects

The pharmaceutical composition for improving ovarian functions and the method of producing the same according to the present invention can be applied to a pharmaceutical composition for relieving or treating early ovarian failure, infertility/subfertility, early menopausal, menopausal and climacteric symptoms by producing placenta-derived mesenchymal stem cells in the form of spheroidal cell aggregates. In particular, compared to monolayer-cultured placenta-derived mesenchymal stem cells, the cell aggregates, specifically spheroidal three-dimensional cultured cell aggregates according to the present invention, have potent therapeutic effects of improving ovarian functions including improving cellular viability during transplantation, enhancing internal estradiol levels and inducing ovarian folliculogenesis.

### DESCRIPTION OF DRAWINGS

The above and other objects, features and other advantages of the present invention will be more clearly understood from the following detailed description taken in conjunction with the accompanying drawings, in which:

FIG. 1 is an image showing a test process to confirm effects of PD-MSC spheroidal cell aggregates on recovery of ovarian functions using a unilaterally ovariectomized (½ ovariectomized) rat model in an embodiment according to the present invention;

FIG. 2 is an image showing the structure of a concave microwell plate applied to the embodiment according to the present invention (q means a diameter).

FIG. 3 shows microscopy results regarding a process of converting Naive PD-MSCs seeded onto the concave microwell plate into PD-MSC spheroidal cell aggregates over time in Example 1.1) of the present invention (Scale bars: 200 μm);

FIG. 4 shows microscopy results regarding samples of PD-MSC spheroidal cell aggregates on the third day in FIG. 3 isolated and harvested from the microwell plate (Scale bar: 200 μm);

FIG. 5 shows scanning electron microscopy (SEM) results regarding PD-MSC spheroidal cell aggregates harvested on the first day in FIG. 3 [Scale bars: 10 nm, arrows mean different kinds of junctions present between adjacent cells];

FIG. 6 shows scanning electron microscopy (SEM) results regarding PD-MSC spheroidal cell aggregates harvested on the third day in FIG. 3 [Scale bars: 10 nm, arrows mean different kinds of junctions present between adjacent cells];

FIG. 7 is a graph showing measurement results regarding diameters of cell aggregates over time described in Example 1.2) according to the present invention;

FIG. 8 is a fluorescence image showing cell viability test results of cell aggregates on the first day of three-dimensional culture described in Example 1.3) according to the present invention (Scale bar: 200 μm);

FIG. 9 is a fluorescence image showing cell viability test results of cell aggregates on the third day of three-dimensional culture described in Example 1.3) according to the present invention (Scale bar: 200 μm);

FIG. 10 is a graph showing normalization of results shown in FIGS. 8 and 9 (means±S.D., n=25);

FIG. 11 is a graph showing measurement results of ratios of ovary weight to body weight in rat models for respective groups both one week and two weeks after transplantation in Example 2.1) according to the present invention;

FIG. 12 shows analysis results of E₂ levels in rat models for respective groups one week and two weeks after transplantation in Example 2.2) according to the present invention;

FIG. 13 shows, one week and two weeks after transplantation, results of real-time PCR analysis performed on human Alu sequences in order to identify whether or not transplantation of PD-MSCs is successful in Example 2 according to the present invention;

FIG. 14 is an immunohistochemical dye image to identify whether or not follicles grow in the ovaries of respective groups in order to confirm effects of PD-MSCs on recovery of ovarian functions in Example 2.3) according to the present invention (Scale bar: 20 nm);

FIG. 15 is an image showing the number of follicles grown one and two weeks after transplantation in order to confirm effects of PD-MSCs on recovery of ovarian functions in Example 2.4) according to the present invention (Scale bar: 20 nm);

FIG. 16 shows analysis results of Nanos3 mRNA expression levels from ovarian tissues by qRT-PCR one and two weeks after transplantation in Example 3.1) according to the present invention;

FIG. 17 shows analysis results of Nobox mRNA expression levels from ovarian tissues by qRT-PCR one and two weeks after transplantation in Example 3.1) according to the present invention;

FIG. 18 shows analysis results of Lhx8 mRNA expression levels from ovarian tissues by qRT-PCR one and two weeks after transplantation in Example 3.1) according to the present invention;

FIG. 19 shows western blotting results of Nanos3 mRNA expression levels from ovarian tissues by qRT-PCR one and two weeks after transplantation in Example 3.2) according to the present invention;

FIG. 20 shows western blotting results of Nobox protein expression levels from ovarian tissues one and two weeks after transplantation in Example 3.2) according to the present invention; and

FIG. 21 shows western blotting results of Lhx8 protein expression levels in Example 3.2) according to the present invention.

### BEST MODE

Hereinafter, the present invention will be described in more detail with reference to embodiments such that those having ordinary knowledge in the field to which the present invention pertains can easily implement the present invention. However, the present invention can be implemented in various forms and is not limited to embodiments described herein.

Hereinafter, if there is no specific mention as to whether the symbol “%” is % by weight or % by volume, the symbol means % by weight and abbreviations used in description of embodiments and drawings have the following meanings unless specifically mentioned otherwise.

PD-MSC spheroidal cell aggregates: PD-MSC spheroids

Monolayer-cultured PD-MSCs (monolayered PD-MSCs, Naive PD-MSCs): Naive PD-MSCs

Unilaterally ovariectomized rats: OVX (ovariectomized) rats

Non-ovariectomized rat group: Control group

Unilaterally ovariectomized rat group (non-transplanted rats, transplantation of culture media as NTx sham controls): NTx group

Group where naive PD-MSCs (5*10⁵ cells) are intravenously injected into unilaterally ovariectomized rats: Naive group

Model wherein 100 PD-MSC spheroidal cell aggregates (about 1*10⁵ cells) are transplanted by injection into the remaining ovary of unilaterally ovariectomized rats: Spheroid group

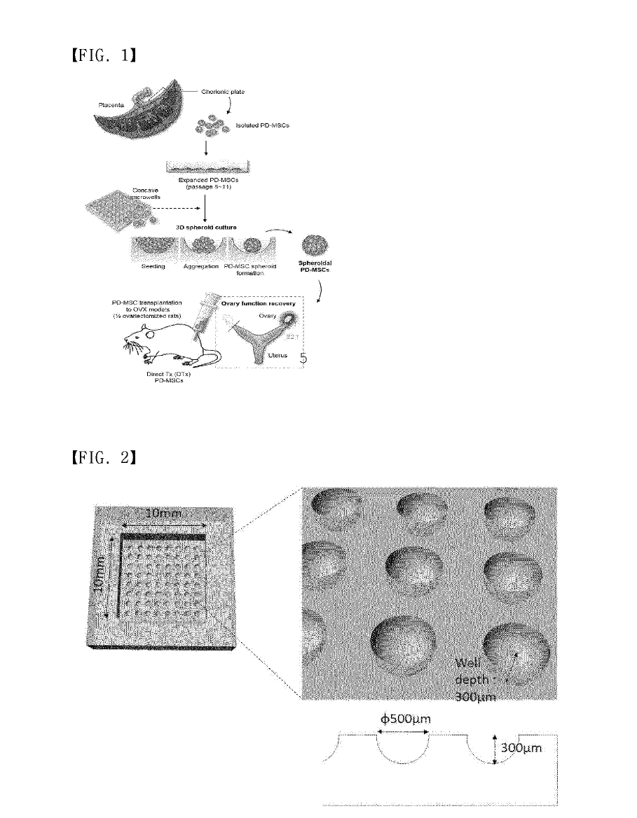

FIG. 1 is an image showing a test process to conform effects of PD-MSC spheroidal cell aggregates on recovery of ovarian functions using a unilaterally ovariectomized rat model described in the following Example. Referring to FIG. 1, mesenchymal stem cells, which were isolated from the chorionic plate of the human placenta, were added to the concave microwell plate and then three-dimensionally cultured, were transplanted into ovariectomized rat models in the form of PD-MSC spheroidal cell aggregates and how PD-MSC spheroidal cell aggregates affect ovarian functions of transplanted rats was identified using the following method.

### <Test Method>

### 1. Preparation of Test Animal

35 6-week-old SD rats (Sprague-Dawley rats, weight of 205 to 215 g) were obtained from Orient Bio Inc., and were bred on a group basis at room temperature before tests. All procedures were carried out in accordance with ethical guidelines provided by Genexine Co. (Seongnam, South Korea).

Unilateral ovariectomy (Ovx; removed the one ovary) was carried out by a method described in research paper (Am J Anat. 1970 January; 127(1):1-7. Effects of unilateral ovariectomy on ovulation and cycle length in 4- and 5-day cycling rats. Peppler R D, Greenwald G S.).

Specifically, ketamine and rompun were mixed at a volume ratio of 3:1 under germfree conditions and rats anesthetized by intramuscular injection at a dose of 100 ul/weight g were subjected to the surgical process for unilateral ovariectomy. The lower part of the back skin of anesthetized rats was shaved and cut to 3 cm to expose the back muscles, muscles were cut to 1 cm and the right ovary was incised, separated and then bound with sterilized sutures to perform unilateral ovariectomy.

Unilaterally ovariectomized rats were allowed to recover for one week.

### 2. Isolation and Culture of Placenta-Derived Mesenchymal Stem Cells

The normal placentas of human having no medical, obstetric or surgical problems were secured. The gestation period was 38 to 40 weeks. All female placentas were provided after written consent relating to placental collection, and the collection and use of the placentas were conducted under the approval of the Institution Review Board (IRB) of Cha hospital.

PD-MSCs (placenta derived mesenchymal stem cells) were isolated in accordance with the method described in the patent (method of isolating high-purity placenta chorionic plate membrane-derived mesenchymal stem cells, Patent No: 10-0900309, grant year: 2009), cultured in a culture dish in a 2D state, and then applied to Naive PD-MSCs.

Specifically, the membrane of the chorionic plate was removed from the placenta, cells were scraped from the membrane and scraped cells were treated with 0.5% collagenase IV (SIGMA) at 37° C. for 30 minutes. The treated cells were cultured in a T25 flask, and transferred at 2×10⁵ cells/cm² to Ham's F-12/DMEM supplemented with 10% FBS (fetal bovine serum) and 1% penicillin-streptomycin, and cultured therein.

### 3. Production of PD-MSC Spheroidal Cell Aggregates

Spheroidal cell aggregates were produced using polydimethylsiloxane (PDMS)-based concave microwell plates.

As shown in FIG. 2, concave microwells with a diameter of 500 μm were produced at a density of 100 wells/cm² (unit of plate) using soft lithography techniques and meniscus of PDMS prepolymers (polydimethylsiloxane prepolymers) described in the registered patent (production of concave microwells using surface tension and formation of cell aggregates using the same, Registration No.: 10-1282926, Registration year: 2013), coated with 3% (w/v) BSA (bovine serum albumin) to prevent cell adhesion and were then applied to the production of spheroidal cell aggregates.

The naive PD-MSCs (8-11 passages) cultured in an ordinary culture dish in a 2D state in 2. above were detached with trysine and then administered at a density of 200,000 cells per plate such that the cells were located inside the cells. Almost all cells were homogeneously mounted 5 minutes after administration and cells which could not be mounted in the concave microwell were removed.

The mounted cells were cultured in a new medium supplemented with 50 ng/ml to 100 ng/ml of FGF-4 and 500 ng/ml to 5 pg/ml of heparin. Cell aggregation and formation of spheroidal cell aggregates were observed with a microscope every day. The diameter of spheroidal cell aggregates was analyzed using Image J software (NIH, Bethesda, Md., USA).

### 4. Evaluation of Viability of PD-MSC Spheroidal Cell Aggregates

In order to identify viability of cells contained in PD-MSC spheroidal cell aggregates (PD-MSC spheroids), PD-MSC spheroidal cell aggregates were cultured in a culture medium in the presence of 50 mM calcein-AM and 25 mg/mL EthD-1 (ethidium homodimer-1; molecular Probes, USA) for 40 minutes at 37° C. and observed with a confocal microscope (Olympus, Japan).

In observation results, green calcein-AM signals mean living cells and red EthD-1 signals mean dead cells. Normalized data of cell viability was obtained from the observation results using ImageJ software.

### 5. Scanning Electron Microscope (SEM) Observation

PD-MSC spheroidal cell aggregates were immobilized in 2.5% glutaraldehyde-containing PBS (phosphate buffered saline) for one hour and softly washed with deionized water 3 to 5 times. For secondary immobilization, spheroidal cell aggregates were immersed in deionized water containing 1% osmium tetroxide for one hour.

The immobilized spheroidal cell aggregates were sequentially immersed in different concentrations of ethanol (25%, 50%, 75%, 95%, and 100%) at room temperature and then immersed in tetrabutyl alcohol for each 30 minutes three times, and then frozen at 70° C. Tetrabutyl alcohol was removed during lyophilization of spheroidal cell aggregates, and the specimens thus produced were coated with a palladium alloy and spheroidal cell aggregates together with graphite paste were observed with a scanning electron microscope (JEOL, Ltd., Tokyo, Japan).

### 6. Transplantation

PD-MSC spheroidal cell aggregates having been cultured for 3 days were harvested from the concave microwell and then used for transplantation. Naive PD-MSCs (5×10⁵ cells) and spheroidal PD-MSCs (1×10⁵ cells) were dyed with a PKH26 Fluorescent Cell Linker Kit (Sigma-Aldrich), and transplanted to the remaining ovary of the rats which were allowed to take a rest for one week after ovariectomy (Naive; n=10, Spheroid; n=10). Culture media as sham controls were transplanted into PD-MSC non-transplanted rats (Non-transplanted rats, NTx; n=10).

The rats of respective groups were sacrificed after one and two weeks, ovarian tissues were harvested and ovary weight and so on was measured from ovarian tissues of all harvested groups (Control, NTx, Naive and Spheroid). Blood samples were harvested from rats every week, and EDTA plasma was isolated by centrifugation, stored at −80° C. and was then used for research. A level of estradiol in plasma was measured in accordance with the manufacturer's instructions using an Estradiol DSL-4400 Radioimmunoassay kit (Diagnostic Systems Laboratories, Inc.).

7. Analysis of qRT-PCR (Quantitative Real-Time Polymerase Chain Reaction)

The DNAs were extracted from the ovary, which had been frozen and then thawed, using phenol/chloroform (SIGMA-Aldrich) and immersed in ethanol and total DNA was then measured based on UN absorbance. Real-time PCR was carried out using 300 ng of a target DNA and an Alu-specific primer with an automation equipment of Applied Biosystems Inc. The target sequence was amplified by repetition of 40 cycles at 95° C. for 2 minutes, at 95° C. for 5 seconds and at 56° C. for 30 seconds and normalized with rat β-actin genes. All reactions were independently repeatedly performed in triplicate.

To check mRNA expression levels of Nanos3, Nobox and Lhx8 relating to oogenesis, quantitative real-time PCT amplification analysis was conducted on mRNA expression levels of markers using an automation machine of Applied Biosystems and SYBR® ExScript™ RT-PCR Kit (TaKaRa). Respective gene expressions were normalized with reference to the GAPDH level as a rat internal reference. The sequence of primers used is shown in the following Table 1. The target sequence was amplified by repeating 40 cycles under temperature conditions including at 95° C. for 10 seconds, at 95° C. for 10 seconds and at 59° C. for 30 seconds. All reactions were independently performed in duplicate.

TABLE 1

| | | Annealing |
|---|---|---|
| Gene | Primer sequences (5′-3′) | temperature (° C.) |
| human Alu | F: CTGGGCGACAGAACGAGATTCTAT | 59 |
| | R: CTCACTACTTGGTGACAGGTTCA | |
| Nanos3 | F: CTC TGC ATG AGG AAG AGG AGC C | 55 |
| | R: GGA CTG ATA GAT CGC ACG AGA | |
| Lhx-8 | F: GTA TCA CTT GGC TTG CTT | 55 |
| | R: ATT ACC GTT CTC CAC TTC | |
| Nobox | F: AGC CAG TGC AGA TCT GCA CC | 50 |
| | R: TGT CAC TGC CAG GAA CAT CCC TC | |
| beta actin | F: TCC TTC TGC ATC CTG TCA GCA | 58 |
| | R: CAG GAG ATG GCC ACT GCC GCA | |
| GAPDH | F: TCC CTC AAG ATT GTC AGC AA | 55 |
| | R: AGA TCC ACA ACG GAT ACA TT | |

### 8. Western Blot Analysis

Ovarian tissues obtained from rats of respective groups were homogenized and dissolved using protein lysis buffer (Sigma-Aldrich). The equivalent amounts of protein lysates obtained from respective rats were harvested from the ovaries at one and two weeks.

Protein lysates were loaded on 10% SDS-PAGE (sodium dodecyl sulfate polyacrylamide gels) and transferred to the PVDF membranes (Bio-Rad Laboratories). The membranes were blocked and then treated at 4° C. with primary antibodies (diluted, 1:1,000) such as anti-LHX8 polyclonal antibody (Santacruz), anti-Nanos3 polyclonal antibody (Abcam) and anti-Nobox polyclonal antibody (Abcam) overnight to induce binding.

After the membranes were washed, reaction proceeded at room temperature for 90 minutes to induce binding of secondary antibodies (horseradish peroxidase conjugated anti-rabbit IgG, 1:10,000, Bio-Rad Laboratories; or anti-goat IgG, 1:5,000, Santa Cruz Biotechnology). After final membranes were washed, target proteins were identified using ChemiDoc (Bio-Rad Laboratories). For normalization of results, all reactions were independently repeatedly performed in triplicate.

### 9. Histological Analysis

To check whether or not ova were mature, the ovary was immersed and immobilized in paraffin, and fragments with a thickness of 3 μm were produced and dyed in accordance with the hematoxylin and eosin (HE) procedure. The fragments were fixed with 4% PFA, dyed with Mayer's hematoxylin, dehydrated with graded ethanol, washed with xylene, and observed using a Zeiss Axioskop2 MAT microscope (Carl Zeiss MicroImaging), and total follicle number was determined for relative normalization.

### 10. Statistical Analysis

All data were expressed as the mean±standard error and were subjected to analysis of two-way ANOVA in accordance with Tukey post-test to examine differences between groups at different times. All statistical analysis was carried out using SPSS software (SPSS) and p value 0.05 was considered statistically significant.

### <Test Results>

### Example 1: Production of Spheroidal Cell Aggregates Using Concave Microwell Plate

As mentioned above, PD-MSC spheroidal cell aggregates were produced in a such way that Naive PD-MSCs were cultured on the PDMS-based concave microwell plate and cells were aggregated.

### 1) Aggregation Process and Surface Observation

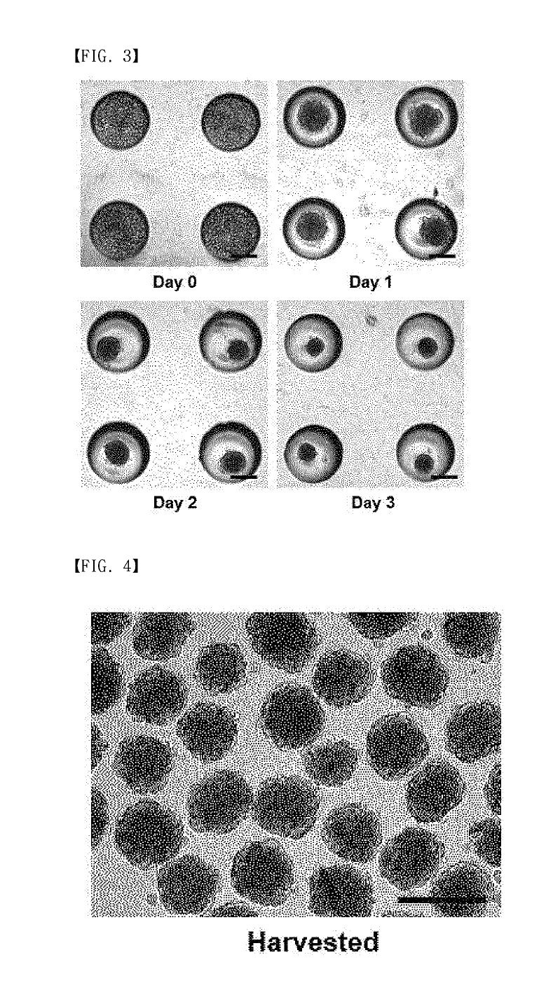

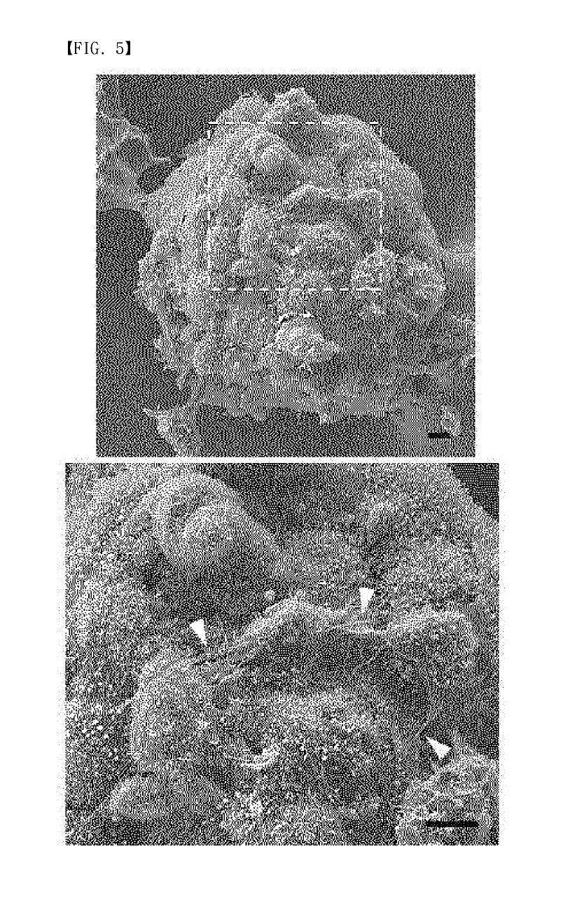

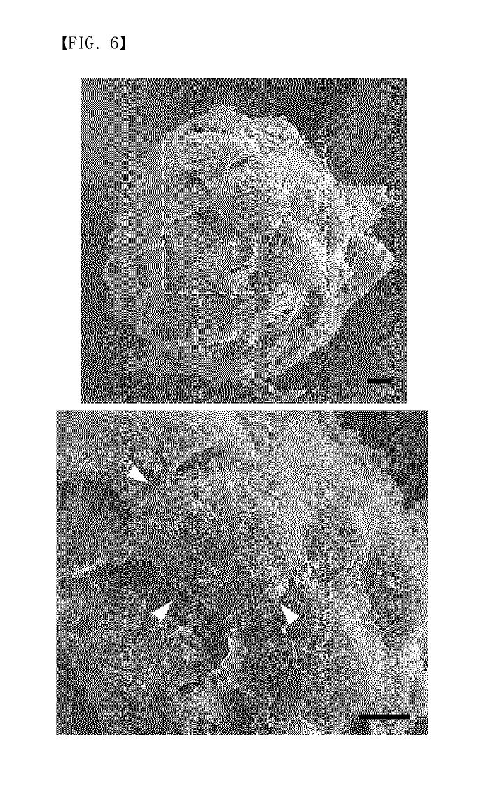

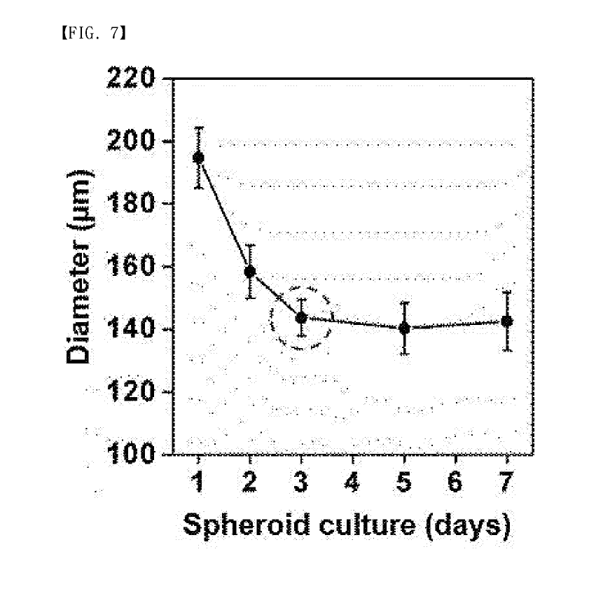

Naive PD-MSCs were observed daily with a microscope starting immediately after administration to 3 days and results are shown in FIG. 3. In addition, SEM images of PD-MSC spheroidal cell aggregates obtained after 3 days are shown in FIG. 4, and SEM images of PD-MSC spheroidal cell aggregates obtained on day 1 and day 3 are shown in FIG. 5 and FIG. 6, respectively.

Referring to FIGS. 4 to 6, PD-MSCs began to aggregate immediately after cells were mounted in the wells and formed spheroidal aggregates after 1 day. Dense spheres with a relatively smooth outer appearance were observed after 2 days (FIG. 3). Spheroidal cell aggregates harvested prior to transplantation had a size controlled by the concave microwells and looked substantially size-homogeneous (FIG. 4).

In order to observe morphological changes which occurred during aggregation of PD-MSCs of three-dimensional culture, the surfaces of PD-MSC spheroidal cell aggregates were observed as SEM images on the first day (Day 1) and the third day (Day 3) after initiating culture (FIGS. 5 and 6).

It could be seen that the spheroidal cell aggregates on the first day had an even surface, whereas cells were stacked relatively flatly on the compact surface on the third day. In particular, magnified SEM images, which are the lower images of FIGS. 5 and 6, showed that the sample on the first day of culture had different shapes of cell-junction complexes (cell-cell junctions) from those of culture sample on the third day.

### 2) Diameter of Cell Aggregates

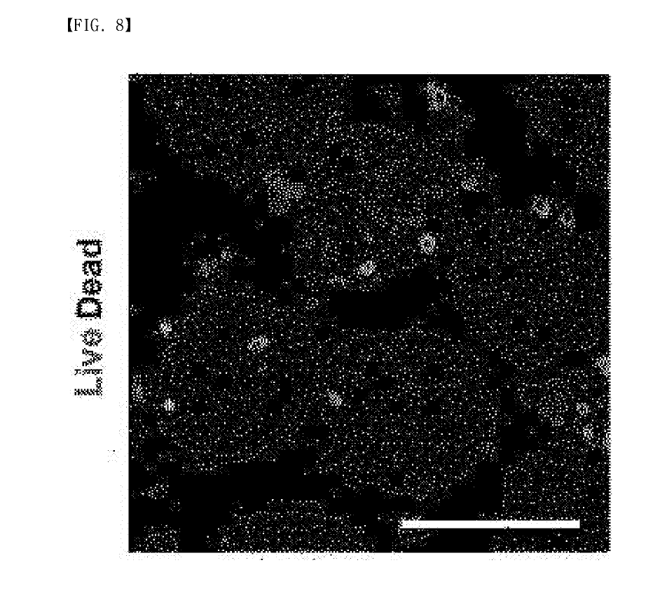

The (mean) diameters of PD-MSC spheroidal cell aggregates cultured in the microwell plate with a diameter of 500 μm, described in the test method above, were measured daily and results are shown as a graph in FIG. 7.

Referring to FIG. 7, the PD-MSC spheroidal cell aggregates on the first day of culture had a mean size of 194.7±9.6 μm, while the PD-MSC spheroidal cell aggregates on the third day of culture had a decreased mean size of 143.7±5.7 μm. It could be seen that the mean size after the third day of culture was substantially maintained to the fifth to seventh days of culture.

### 3) Evaluation of Cell Viability of Cell Aggregates

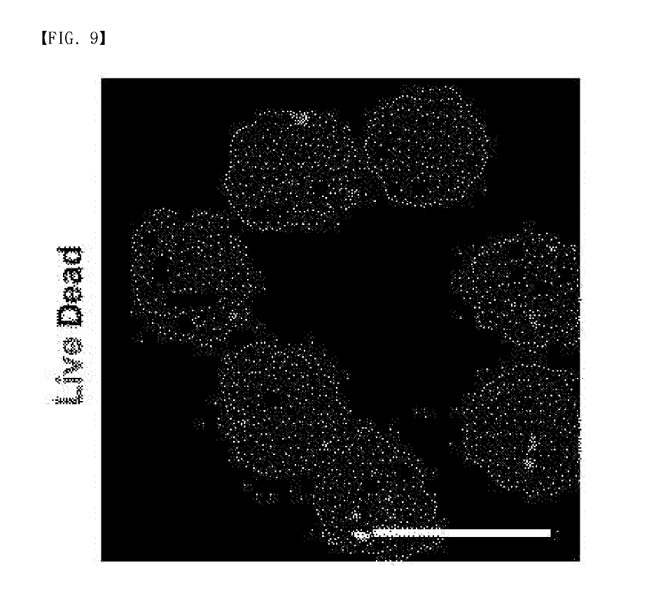

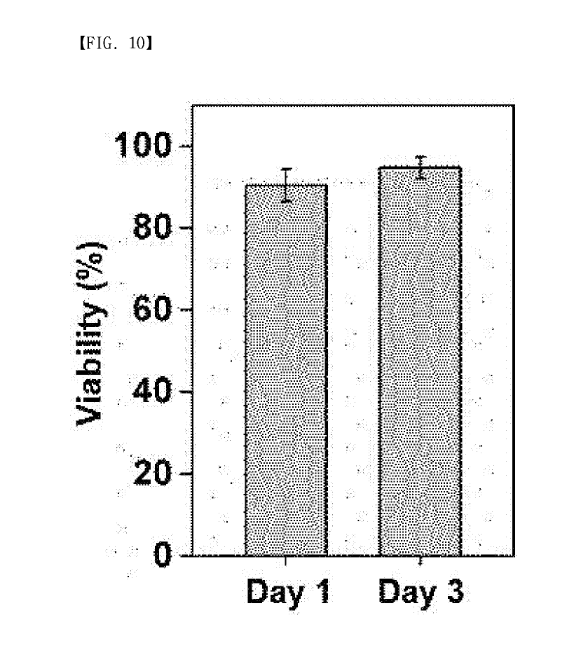

Cell activity of PD-MSC spheroidal cell aggregates was evaluated on the first day of culture and the third day of culture. Fluorescence images of PD-MSC spheroids dyed with live/dead assay reagents are shown in FIG. 8 (1^{st} day) and FIG. 9 (3^{rd} day) and normalization of these results is shown in FIG. 10.

Referring to FIGS. 8 and 9, fluorescence images of green living cells and red dyed cells showed that debris present on the surfaces of cell aggregates disappeared over time and almost all cells including spheroidal cell aggregates were green, which means that the cells had a considerably high cell viability.

As can be seen from FIG. 10 showing normalization of these results, cell viability was high both on the first day of culture and the third day of culture(respectively, 90.4±3.9% and 94.8±2.7%), which means that placenta-derived stem cell spheroidal cell aggregates were effectively cultured using PDMS concave microwell plates.

### Example 2: Tests for Effects of PD-MSC Spheroidal Cell Aggregates Transplanted to Ovariectomized Rats on Recovery of Ovarian Functions

### 1) Ovary Weight Analysis

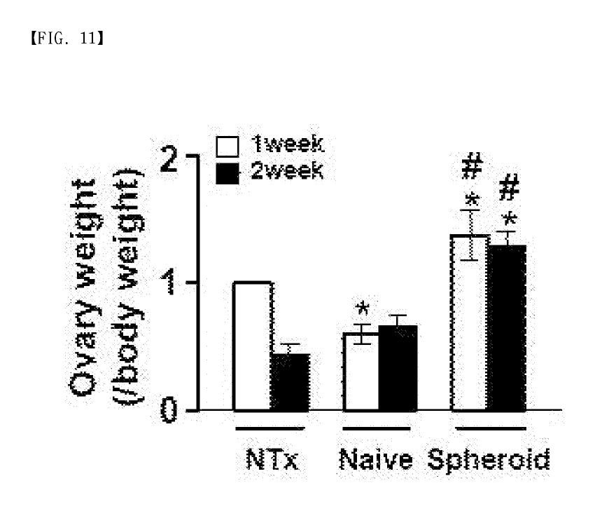

In order to identify effects of PD-MSC spheroidal cell aggregates transplanted to ovariectomized rat models, the body weight of rat models and the ovary weight of the corresponding rat models were measured 1 week and 2 weeks after transplantation, a ratio of ovary weight to body weight was calculated and results are shown in FIG. 11.

Referring to FIG. 11, the ovary weight of the Naive group was decreased one week after transplantation (p<0.05). Ovary weights of spheroids groups one and two weeks after transplantation were considerably increased, compared to the NTx group (p<0.05). Furthermore, one and two weeks after transplantation, ovary weights of spheroid groups were considerably increased, compared even to ovary weight of the Naive group (p<0.05).

### 2) Serum Estradiol Level Analysis

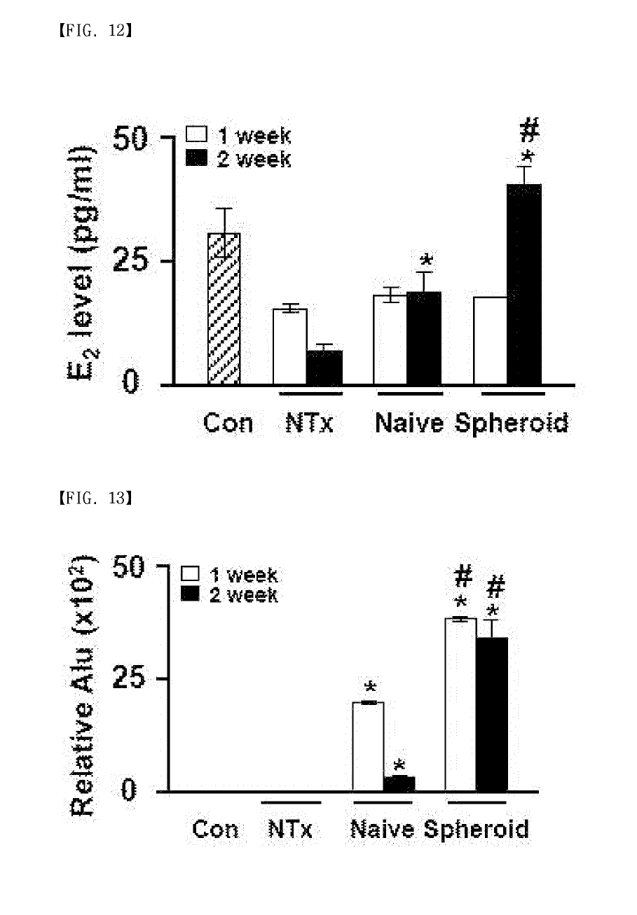

In order to study effects of PD-MSC transplantation on ovarian functions, serum estradiol levels (E₂ levels) for respective groups were analyzed and the results are shown in FIG. 12.

Referring to FIG. 12, one week after transplantation, the NTx group had an about 50% decrease in E₂ level, compared to the control group (30±4.8 pg/mL vs. 15.58±0.76 pg/mL; p<0.05). In addition, the E₂ level of the NTx group was rapidly decreased one week after transplantation and had an about 77% decrease two weeks, compared to the control group (6.9±1.4 pg/mL; p<0.05).

Two groups, to which PD-MSCs were transplanted, (Naive group and Spheroids group) showed an increase in E₂ level 2 weeks after transplantation, compared to the NTx group (18.9±3.8 pg/ml; Naive group, 40.3±3.8 pg/ml; Spheroid group, p<0.05). In addition, 2 weeks after transplantation, the E₂ level of Spheroid group was remarkably higher than that of the Naive group (p<0.05).

### 3) Analysis of Human Alu Sequences

In order to identify whether or not transplantation of PD-MSCs was successful, real-time PCR analysis was performed on human Alu sequences and results are shown in FIG. 13.

As can be seen from the results shown in FIG. 13, human Alu sequences were not observed in the Control group and the NTx group, whereas human Alu sequences were significant levels only in transplanted groups (Naive group and Spheroid group) (p<0.05).

Expression of the Alu sequence in the ovary of Spheroid group continuously maintained its increased level (p<0.05), whereas expression of the Alu sequence in the ovary of Naive group was rapidly decreased 2 weeks after transplantation, compared to one week after transplantation.

This proves that, Spheroid group, where PD-MSCs were transplanted in the form of cell aggregates, was advantageous for cell survival in vivo, compared to other groups and functions thereof can be continuously maintained.

### 4) Analysis of Total Number of Follicles in Ovary

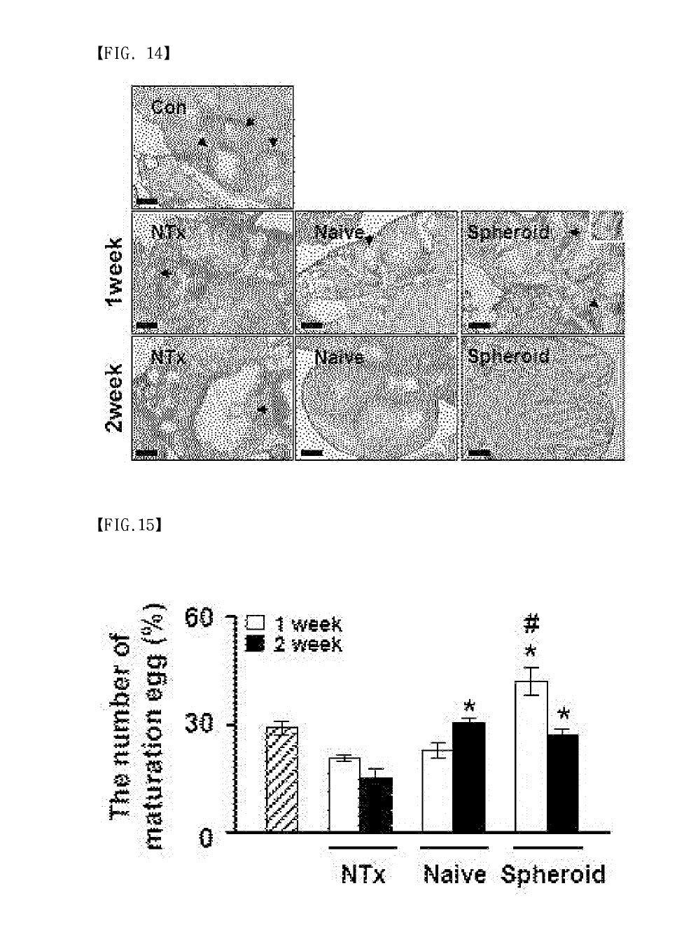

In order to identify effects of PD-MSCs on recovery of ovarian functions, growth and number of follicles were observed in the ovary of all groups and results are shown in FIGS. 14 and 15.

As can be seen from the results shown in FIGS. 14 and 15, the number of follicles of NTx group was decreased compared to Control group (p<0.05). The number of follicles of Naive group was similar to that of NTx group one week after transplantation, while the number of follicles of Naive group was doubled compared to that of NTx group, two weeks after transplantation (30.3±1.2 vs. 15±2.5, p<0.05). Spheroid group-applied samples had almost double the number of follicles as NTx group and this behavior has been maintained, regardless of the number of weeks, since 2 weeks of transplantation, (p<0.05).

Based on one week after transplantation, the number of follicles of Spheroid group was about 1.8 times that of Naive group (p<0.05).

This result means that PD-MSCs directly transplanted to the ovary can recover ovarian functions by controlling estradiol production after ovariectomy. In addition, this proves that cell aggregates have better therapeutic effects than Naive PD-MSCs.

### Example 3: Evaluation of Ovarian Folliculogenesis Activity of PD-MSC Spheroidal Cell Aggregates Transplanted to Ovariectomized Rat

As factors affecting formation of ovarian follicles, Nanos3, Nobox (Newborn ovary homeobox) and Lhx8 (LIM-homeobox protein8) and the like are known. Expression of ovarian folliculogenesis markers was tested by real-time PCR and western blotting in the ovary of ovariectomized rats, to identify effects of PD-MSCs transplantation on ovarian folliculogenesis.

1) qRT-PCR Analysis

mRNA expression levels of Nanos3 (Nanos), Nobox and Lhx8 were analyzed from ovarian tissues by qRT-PCR and the results are shown in FIGS. 16 to 18.

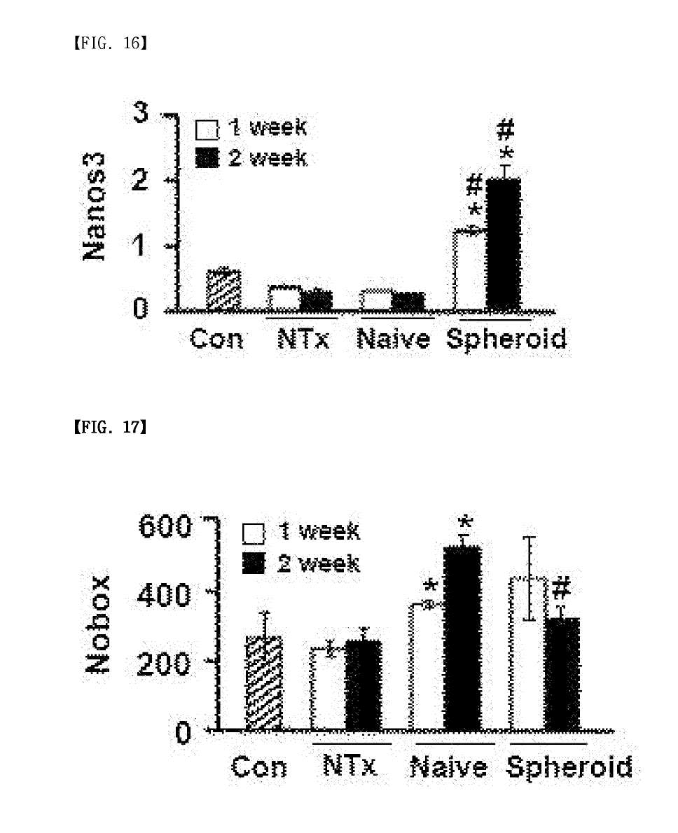

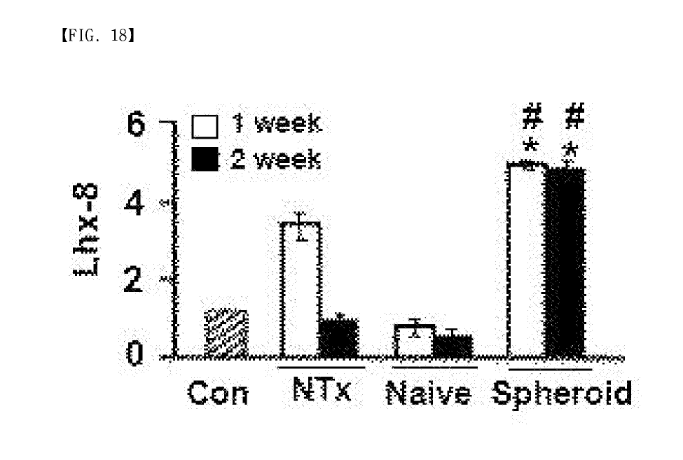

In the 1^{st} week and the 2^{nd} week, Naive group had a significantly increase in mRNA expression levels of Nobox, compared to NTx group (p<0.05), and Spheroid group showed no significant difference (see FIG. 17). However, both in one week and two weeks after transplantation, Spheroid group showed a significant increase in mRNA expression of Nanos and Lhx8, compared to NTx group and Naive group (p<0.05, see FIGS. 16 and 18).

### 2) Western Blot Analysis

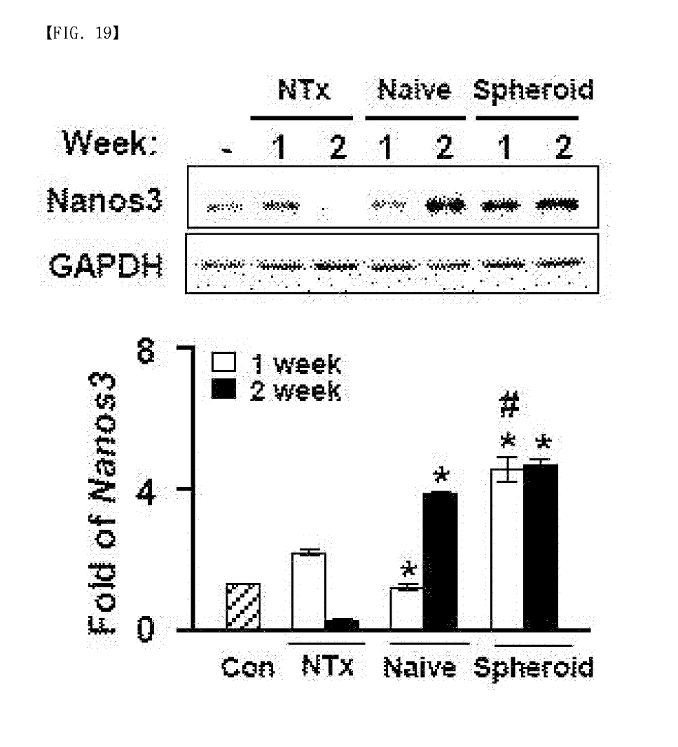

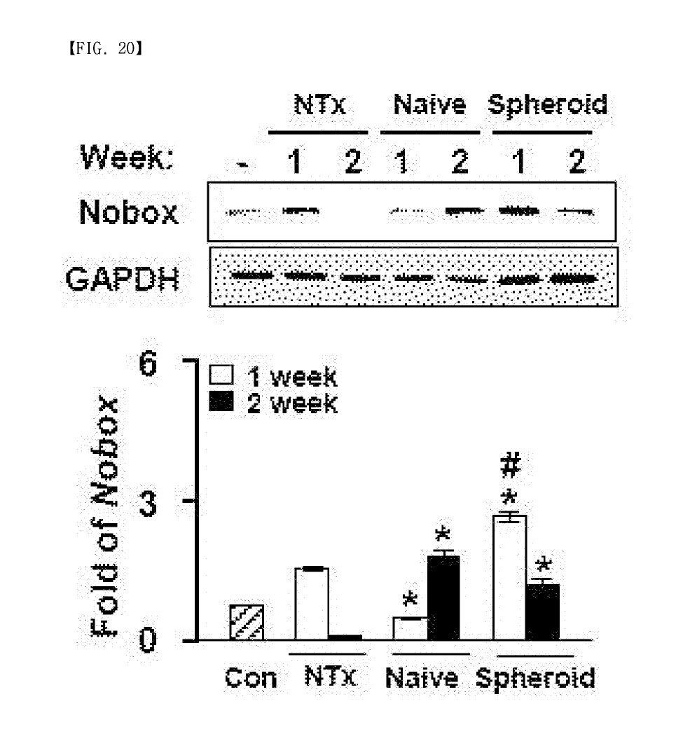

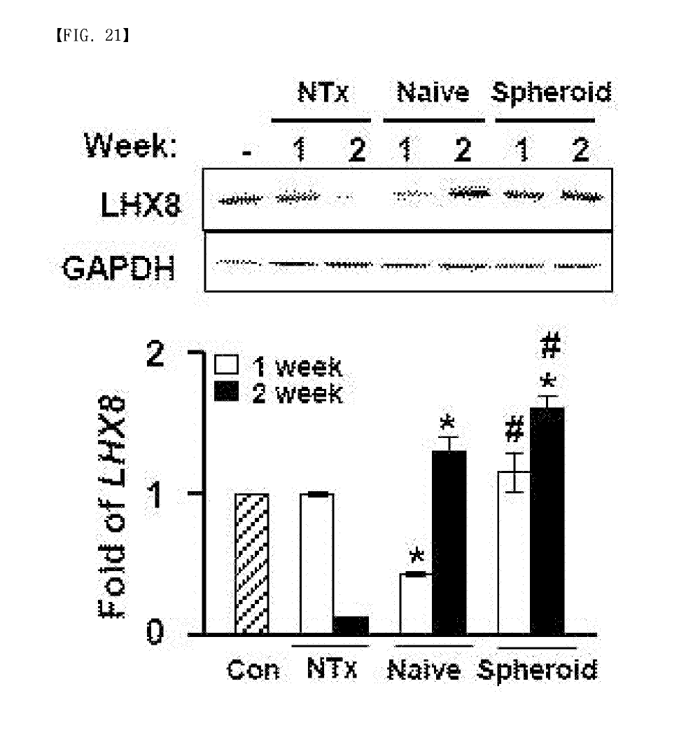

Western blotting results indicating expression levels of Nanos3, Nobox and Lhx8 proteins are shown in FIGS. 19 to 21.

As can be seen from western blotting results, Naive group showed increases in expression of Nanos3, Nobox and Lhx8 proteins, compared to NTx group, two weeks after transplantation (p<0.05). In addition, Spheroid group showed significant increases in expression of Nanos3, Nobox and Lhx8 proteins, compared to NTx group, both one week and two weeks after transplantation (p<0.05).

Spheroid group showed significant increases in expression of Nanos3 and Nobox proteins, one week after transplantation, compared to Naive group (p<0.05). Spheroid group showed a rapid increase in expression of Lhx8, both one week and 2 weeks after transplantation, compared to Naive group (p<0.05).

As can be seen from the results, expression levels of factors, which affect formation of ovarian follicles, were significantly increased by transplantation of PD-MSCs. Taking this point into consideration, transplantation of PD-MSCs promotes ovarian folliculogenesis.

The test results described above showed that PD-MSC spheroidal cell aggregates transplanted into Spheroid group can improve ovarian functions after transplantation and, in consideration of differences in gene expression after cell transplantation, variations in E₂ concentrations and maturity levels of follicles, in 1 week after transplantation, the transplanted PD-MSC spheroidal cell aggregates directly contribute to the improvement of ovarian functions and these functions can be maintained.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

Funding Sources:

This research was supported by the Bio & Medical Technology Development Program of the National Research Foundation (NRF)& funded by the Korean government (MSIT) (NRF-2017M3A9B406172).

### INDUSTRIAL APPLICABILITY

As is apparent from the above description, the pharmaceutical composition for improving ovarian functions according to the present invention and a method of preparing the same can be applied to a pharmaceutical composition for relieving or treating early ovarian failure, infertility/subfertility, premature menopausal, menopausal and climacteric symptoms, a method of preparing the same, and a method of treating the same.

1. A pharmaceutical composition for improving ovarian functions comprising spheroidal cell aggregates including placenta-derived mesenchymal stem cells as an active ingredient.

2. The pharmaceutical composition according to claim 1, wherein the spheroidal cell aggregates have less junction boundary between cells due to junctional complexes between adjacent cells and extracellular matrices (ECMs), and thus a relatively smooth outer surface.

3. The pharmaceutical composition according to claim 1, wherein the pharmaceutical composition for improving ovarian functions is used to relieve or treat at least one symptom selected from the group consisting of early ovarian failure, infertility, subfertility, early menopausal, menopausal and climacteric symptoms.

4. The pharmaceutical composition according to claim 1, further comprising an adjuvant.

5. The pharmaceutical composition according to claim 1, wherein the pharmaceutical composition is formulated in the form of an injection.

6. A use for preparing a pharmaceutical composition for improving ovarian functions comprising spheroidal cell aggregates including placenta-derived mesenchymal stem cells.

7. The use according to claim 6, wherein the spheroidal cell aggregates are obtained by three-dimensionally culturing placenta-derived mesenchymal stem cells, and have less junction boundary between cells due to junctional complexes formed between adjacent cells and extracellular matrices (ECMs), and thus a smooth outer surface, compared to placenta-derived mesenchymal stem cells before culture.

8. The use according to claim 6, wherein the spheroidal cell aggregates have a mean diameter of 250 μm or less.

9. A method of producing spheroidal cell aggregates for improving ovarian functions comprising:
adding placenta-derived mesenchymal stem cells to a concave microwell plate;
culturing the placenta-derived mesenchymal stem cells in the microwells using a medium for culturing cell aggregates containing a cell growth factor, to create spheroidal cell aggregates including placenta-derived mesenchymal stem cells; and
harvesting the spheroidal cell aggregates.

10. A method of preventing or treating a disease relating to deterioration in ovarian functions comprising administering, to a mammal, a pharmaceutical composition for improving ovarian functions comprising spheroidal cell aggregates including placenta-derived mesenchymal stem cells.
